(19) United States
(12) Patent Application Publication
(10) Pub. No.: US 2006/0288429 A1
(43) Pub. Date: Dec. 21, 2006

(54) Genetic screen in drosophila for metastatic behavior

(75) Inventors: Tian Xu, Guilford, CT (US); Raymond Pagliarini, Baltimore, MD (US); Tatsushi Igaki, New Haven, CT (US)

Correspondence Address: FISH & NEAVE IP GROUP;ROPES & GRAY LLP, ONE INTERNATIONAL PLACE, BOSTON, MA, 02110-2624, US

(73) Assignee: Yale University, New Haven, CT (US)

(21) Appl. No.: 11/399,880

(22) Filed: Apr. 7, 2006

Related U.S. Application Data
(63) Continuation of application No. PCT/US04/33310, filed on Oct. 7, 2004.
(60) Provisional application No. 60/509,396, filed on Oct. 7, 2003.
(60) Provisional application No. 60/611,944, filed on Sep. 21, 2004.

(51) Int. Cl. A01K 67/033; A01K 67/027

(52) U.S. Cl. 800/3; 800/13; 800/18

(57) ABSTRACT

The disclosure provides, among other things, assays for identifying compounds and genes that affect metastatic behavior of cells.

### RELATED APPLICATIONS

This application claims the benefit of the filing date of U.S. Provisional Application No. 60/509,396, filed Oct. 7, 2003, entitled “Genetic screen in drosophila for metastatic behavior”, by Tian Xu and Raymond Pagliarini and U.S. Provisional Application No. (Serial No. not assigned), filed Sep. 21, 2004, entitled “Loss of cell polarity drives tumor growth and metastasis through JNK activation in Drosophila”, by Tian Xu. The teachings of the referenced applications are incorporated by reference herein in their entirety.

### STATEMENT REGARDING FEDERAL FUNDING

Work described herein was funded, in whole or in part, by NIH grant CA69408. The United States government has certain rights in the invention.

### BACKGROUND

Metastasis is the most common cause of cancer fatalities (Chambers et al., 2002). While our knowledge of cancer initiation has improved with the identification of various oncogenes and tumor suppressor genes, the identification of analogous genes that promote tumor progression and metastasis when mutated has not been generally successful (Bernards and Weinberg, 2002). This underscores the importance for new model systems for identifying new candidate genes involved in metastasis and dissecting their function.

Thus, there is a need for improved methods for identifying genes involved in metastatic tumor growth and improved methods for identifying anti-metastatic agents.

### SUMMARY

This disclosure provides, in part, novel systems for identifying genetic alterations and compounds that affect metastasis. Drosophila provide a genetic model to study cancer biology. The biology of epithelial cells, the tissue type most commonly mutated in human cancer, is remarkably similar in flies and humans. In developing Drosophila larvae, the imaginal discs that go on to form adult tissues proliferate with a cell cycle analogous to humans. Moreover, many genes involved in essential biological processes such as cell proliferation, cell growth, cell survival, and cell death are conserved between flies and humans. Indeed, alterations in some of these genes, such as inactivation of lats or activation of Ras, have been shown to produce overgrowths and tumors in both flies and mammals (Hingorani et al., 2003; Karim and Rubin, 1998; Tao et al., 1999; Xu et al., 1995). Another advantage of the fly system for cancer biology is the use of genetic mosaic analysis, which mimics the clonal development of human cancers and allows for the discovery of novel tumor suppressor genes (Xu and Harrison, 1994; Xu and Rubin, 1993). The present disclosure teaches a variety of methods for use with transgenic flies and further provides evidence that information obtained from flies is readily transferable to mammalian systems.

In certain aspects, the disclosure provides a method for identifying a mutation that induces metastatic behavior in cells. “Metastatic behavior” may be understood generally as the tendency of cells to invade neighboring or distant tissues, and may also be evidenced by increased motility, changes in cellular morphology associated with invasiveness and loss of adhesion to neighboring cells. Metastatic behavior may be assessed by reference to a suitable control, typically an animal having cells of the same genotype but not subjected to the perturbation (e.g., not having the test mutation). A method disclosed herein may comprise: (a) providing a transgenic non-human animal in which cells comprise (i) a genotype that induces non-invasive tumor formation and, (ii) a candidate mutation; and (b) evaluating a metastatic behavior in the animal, wherein an increase in the metastatic behavior of the cells in the animal relative to a suitable control indicates that the test mutation increases metastatic behavior. The genotype that induces the formation of a noninvasive tumor may be restricted to a subset of cells in the animal. This may be achieved by employing a recombinase system such as FLP/Frt and Cre/lox, particularly where the recombinase is controlled by tissue-selective, developmentally-regulated or conditional promoters, and in addition, may be used to control the cells in which a particular genotype occurs. The genotype that induces noninvasive tumor formation may be, for example, nucleic acid encoding an oncogene and a loss of function mutation in a tumor suppressor gene. Often a tumorigenic effect of the loss of function mutation in the tumor suppressor gene will occur only in the absence of any functional copy of the tumor suppressor gene (e.g., a homozygote for loss of function). An example of a suitable oncogene is oncogenic Ras, such as the Ras^{V12} mutation. An example of tumor suppressor gene is the lats gene. Optionally, the transgenic animal is a transgenic fly, such as Drosophila or a transgenic non-human mammal, such as a transgenic mouse.

In certain aspects, the disclosure provides methods for identifying a mutation that induces metastatic behavior in human cells. Such a method may comprise (a) providing human cells which comprise: (i) oncogenic Ras or a tumor suppressor loss of function mutation, and (ii) a candidate mutation; and (b) evaluating a metastatic behavior in the cells, wherein an increase in the metastatic behavior of the cells relative to a suitable control indicates that the test mutation increases metastatic behavior. Detection of a metastatic behavior may include, for example, transplanting the human cells into a suitable animal host (e.g. mouse) and determining whether metastasis occurs, wherein if metastasis occurs, a mutation that induces metastatic behavior has been identified. Metastatic behavior may also be assessed by evaluating features such as cell motility or migration in vivo or in vitro, cell-cell adhesion, cell morphology, and the like.

In certain aspects, the disclosure provides a non-human transgenic animal that develops metastatic tumors. Such an animal may include cells that comprise: (a) an oncogene or tumor suppressor loss of function mutation that induces non-invasive tumor formation and, (b) a loss of function mutation in a cell polarity determining gene. The oncogene or loss of function mutation may occur in only a subset of cells in the animal. Expression of an oncogene may be controlled by, e.g., expression from a regulated promoter. Genotypes may be restricted to certain cell types by use of recombinase systems. Preferably, the oncogene is Ras, particularly Ras^{V12}. The animal may be any genetically tractable animal, but will preferably be a mouse or a fly. Cell polarity genes are those genes that are generally involved in maintaining polarity of epithelial cells, and may also have an effect on the morphology of epithelial cell monolayers. Examples of the cell polarity determining genes include the scribble family [scribble (D. melanogaster), Scrib (human), Scrib1 (mouse)], the Lethal giant larvae (Lgl) family [Lgl (D. melanogaster), Hug1 (human), Lgl1 (mouse)], the Discs large family [Dlg (Drosophila, mouse, human)], the Cdc42 family [Cdc42 (Drosophila, human, mouse)], the bazooka family and the stardust family. Any recognizable homolog of the preceding may be used.

In certain aspects, animals described herein may be used in a screening method for identifying inhibitors of metastatic tumor growth. Such a method may comprise: (a) administering a candidate inhibitor to a test transgenic animal disclosed herein having cells that exhibit a metastatic behavior, and (b) evaluating metastatic behavior of cells in the test animal, wherein a decrease in metastatic behavior of cells in the test transgenic animal indicates that the candidate inhibitor is an inhibitor of metastatic behavior in cells.

In certain aspects, the disclosure provides a method of screening for an inhibitor of metastatic behavior in cells. Such a method may comprise: (a) administering a candidate inhibitor to metastatic cells, wherein the metastatic cells comprise: (i) an oncogene that induces non-invasive tumor formation and, (ii) a loss of function mutation in a cell polarity determining gene; and (b) evaluating a metastatic behavior in the metastatic cells, wherein a decrease in the metastatic behavior of the cells relative to a suitable control indicates that the candidate inhibitor decreases metastatic behavior in cells.

In certain aspects, xenotransplant methods may be used to identify inhibitors of metastatic behavior in cells. A method may comprise: (a) providing a non-human animal comprising transplanted human metastatic cells, wherein the metastatic cells comprise: (i) an oncogene that induces non-invasive tumor formation and, (ii) a loss of function mutation in a cell polarity determining gene; (b) administering a candidate inhibitor to the non-human animal; and (c) evaluating a metastatic behavior in the cells, wherein a decrease in the metastatic behavior of the cells relative to a suitable control indicates that the candidate inhibitor decreases metastatic behavior. Preferably, the non-human animal will be a mouse.

In certain aspects, the present disclosure provides the identity of genes and proteins involved in metastasis. Such genes may be targets for therapeutic intervention. A cancer patient may be treated with an agent that inhibits or activates such a protein, as appropriate, to inhibit metastatic growth. Examples of the identified genes include those of the JNK pathway, the activity of which is demonstrated herein to be needed for metastatic behavior in cells carrying a Ras oncogene. The JNK pathway include JNKKK, JNKK and JNK proteins, as well as the upstream TNF-alpha signaling pathway, including TNF-alpha, TNFR and the TRAF proteins, particularly TRAF2. In certain aspects, the disclosure provides a method of identifying an agent that may be used to inhibit metastatic cancer growth in a subject, the method comprising: (a) identifying an agent that inhibits JNK pathway activation; and (b) evaluating the effect of the agent on a metastatic behavior of a metastatic cell, wherein an inhibitor of JNK pathway activation that inhibits metastatic behavior of a metastatic cell is an agent that may be used to inhibit metastatic cancer growth in a subject. JNK activity, or activity of another kinase in the pathway, may be assessed in biochemical or cell-based assays by determining phosphorylation of an appropriate substrate, such as Jun in the case of JNK. JNK pathway activity may also be assessed by determining expression of a nucleic acid, preferably a nucleic acid encoding a reporter gene, that is under control of a promoter that is responsive to JNK, such a Jun regulated promoter. In a preferred embodiment, an agent is identified that inhibits TAK1 kinase of human, mouse or Drosophila. Identifying an agent that inhibits JNK pathway activation may comprise identifying an agent that inhibits TNF-alpha signaling.

Cell polarity genes and proteins are also identified herein as affecting metastatic behavior of cells. As demonstrated here, loss of function mutations in these genes synergize with Ras oncogenes to generate metastatic behavior. Accordingly, it is expected that activators or agonists of cell polarity genes will have anti-metastatic activity. In certain aspects, the disclosure provides a method of identifying an agent that may be used to inhibit metastatic cancer growth in a subject, the method comprising: (a) identifying an agent that activates a cell polarity protein; (b) evaluating the effect of the agent on a metastatic behavior of a metastatic cell, wherein an activator of a cell polarity protein that inhibits metastatic behavior of a metastatic cell is an agent that may be used to inhibit metastatic cancer growth in a subject. The cell polarity protein may be selected from the group consisting of: scribble (D. melanogaster), Scrib (human), Scrib1 (mouse), Lgl (D. melanogaster), Hub1 (human), Lgl1 (mouse), Dlg (Drosophila, mouse, human), Cdc42 (Drosophila, human, mouse), bazooka, stardust, cdc42 and another mammalian homolog of any of the preceding.

The E-cadherin gene and protein, and other members of the E-cadherin signaling pathway, such as beta-catenin, are also identified herein as affecting metastatic behavior of cells. As demonstrated here, loss of function mutations in these genes synergize with JNK activation and Ras oncogenes to generate metastatic behavior. Accordingly, it is expected that activators or agonists of E-cadherin and its pathway will have anti-metastatic activity. In certain aspects, the disclosure provides a method of identifying an agent that may be used to inhibit metastatic cancer growth in a subject, the method comprising: (a) identifying an agent that activates an E-cadherin pathway protein; (b) evaluating the effect of the agent on a metastatic behavior of a metastatic cell, wherein an activator of the E-cadherin pathway that inhibits metastatic behavior of a metastatic cell is an agent that may be used to inhibit metastatic cancer growth in a subject. Activating an E-cadherin pathway may comprise activating or upregulating a protein selected from the group consisting of: E-cadherin, armadillo, beta-catenin and another mammalian homolog of any of the preceding.

### BRIEF DESCRIPTION OF THE DRAWINGS

FIG. 1. A Drosophila genetic model for studying tumor progression. (A) Clones of GFP-labeled cells in the eye disc carry a genetic alteration that either activates an oncogene or inactivates a tumor suppressor gene. This results in localized noninvasive tumors that never move from the head region (magnification, x40). Second-site genetic alterations may cause the development of metastatic behaviors, resulting in the presence of GFP-marked cells in ectopic sites. (B) Expression of the FLP recombinase in the developing eye [eyFLP (6)] mediates mitotic recombination between chromosome arms (34) and produces clones of cells homozygously mutant for a gene that promotes metastatic behavior (i.e., scrib). Only these mutant cells lose the Gal80 repressor (35), which allows Gal4 [under control of the eyFLP-activated Act>y⁺>Gal4 “flip-out” construct (36)] to direct the constitutive expression of UAS-GFP and UAS-Ras^{V12} [as well as other genes of interest (37)] regardless of their eventual locations and differentiation status. Gal80 expression in nonmutant cells also markedly reduces leaky flip-out construct expression in tissues outside of the eye-antennal region. Expression of GFP, Ras^{V12}, and other transgenes is therefore restricted to homozygous mutant cells. Multiple genetic alterations can be combined in the same cell and metastatic behavior can be monitored in vivo by following these GFP-expressing cells.

FIG. 2. A model for cooperative induction of enhanced tumor growth and metastasis by JNK and oncogenic Ras in Drosophila. (A) Ras^{V12}-expressing noninvasive tumors show a modest growth advantage with moderate cell proliferation and cell growth. (B) Loss of cell polarity alone triggers JNK pathway activation through DTRAF2 and dTAK1, resulting in cell death. (C) Loss of cell polarity in Ras^{V12}-expressing tumors results in an enhanced tumor growth through the cooperation between Ras and JNK signaling; the pro-apoptotic effect of JNK signaling is converted to a pro-tumor effect in the presence of Ras signaling. JNK and Ras^{V12} also cooperate with each other to induce tumor metastasis in conjunction with a loss of E-cadherin/catenin complex.

### DETAILED DESCRIPTION

The application will be more readily understood by reference to the following examples, which are included merely for purposes of illustration of certain aspects and embodiments of the present application, and are not intended to limit the application.

### EXAMPLE 1

### Establishment of a Genetic Screen in Drosophila for Metastatic Behavior

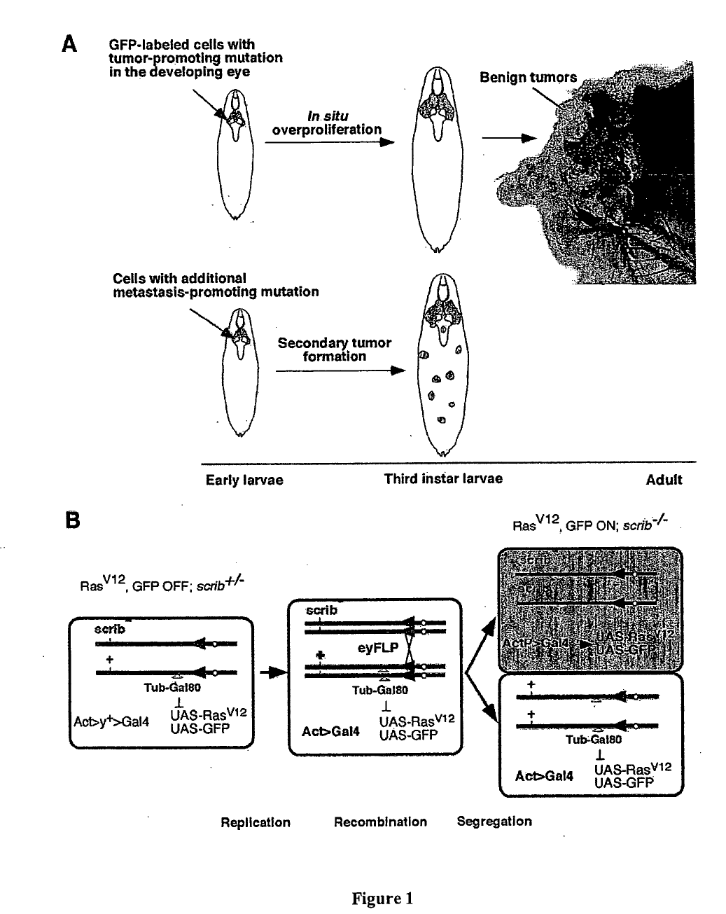

To develop a Drosophila metastasis model, we wished to (i) induce noninvasive tumors in a defined location, through either expression of an oncogene or inactivation of a tumor suppressor gene; (ii) genetically label these tumor cells with a visible marker such as green fluorescent protein (GFP); and (iii) explore whether additional genetic alterations in these cells could elicit metastatic behavior (e.g., the movement of GFP-labeled tumor cells into different tissues) (FIG. 1A). Our genetic scheme used eyeless promoter-driven FLP recombinase expression (eyFLP) (FIG. 1B) (5). This allowed the introduction of multiple genetic alterations (either loss-of-function mutations or gene overexpression) into GFP-labeled cells specifically in the developing larval eye-antennal imaginal discs. Because tumor cells were genetically produced, mechanical disruption of cells and extracellular matrix was avoided. Also, tumor progression and metastatic behavior could be easily monitored and studied in living flies because GFP-labeled tumor cells could be observed in transparent larvae.

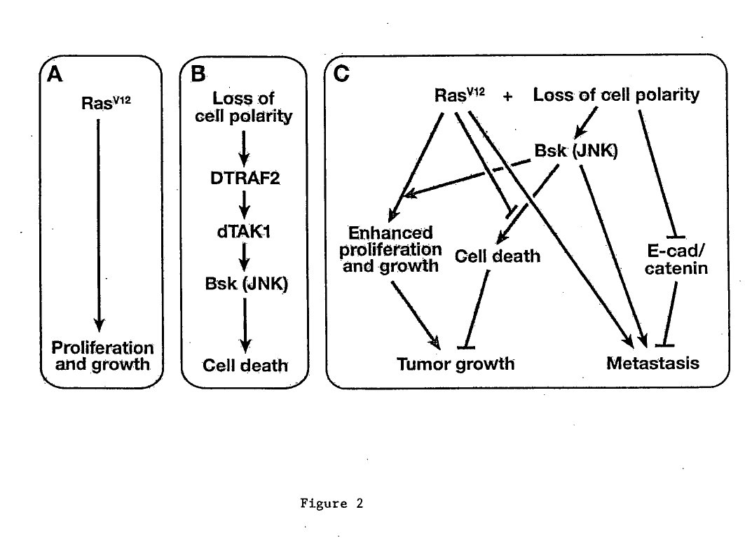

Clones of GFP-labeled wild-type cells were analyzed in the whole bodies of third-instar larvae, pupae, and adults (FIG. 2A; n>1000). GFP was observed in the larval eye-antennal imaginal discs as well as in the optic lobes of the brain, but was not detected in other adjacent tissues such as the ventral nerve cord (VNC) (ey also expresses in the CNS, ocelli, and Bolwig's organ; in larvae, however, eyFLP did not produce visible clones outside of the noted locations). GFP was also observed in other tissues and occurred in reproducible locations, depending on the particular eyFLP transgene used [mostly in the gonad].

Alterations in the Ras oncogene or the lats tumor suppressor gene contribute to tumorigenesis in both flies and mammals (6-9). We generated flies with noninvasive tumors by inducing clones of cells either mutant for lats or expressing activated Ras (UAS-Ras85D^{G12V} or “Ras^{V12}”; n>1000 for each). In either case, the amount of GFP-marked tissue in the eye-antennal discs was noticeably increased, and most mutant flies died before adulthood. However, GFP-labeled mutant cells were always located in the same areas as in the wild type. Mutant cells were not seen outside of the eye-antennal disc/optic lobe region even after eye disc eversion, suggesting that tissue integrity was not compromised. Thus, this system allowed the potential detection of metastatic behaviors caused by additional mutations.

We used the GFP-labeled Ras^{V12} eye expression system to screen for additional preexisting or newly induced mutations that promoted tumor progression. Three phenotypic classes were observed when Ras^{V12}expressing cells in mosaic flies were also made homozygous for additional second-site mutations (862 mutant lines): reduced tumor growth (76 lines), enhanced tumor growth (9 lines), and tumors with metastatic behavior (2 lines). Mutations in the scrib gene (10) in conjunction with Ras^{V12} expression (heretofore referred to as Ras^{V12}/scrib^{-/-} ) caused metastatic behavior. Third-instar Ras^{V12}/scrib^{-/-} larvae carried large primary tumors and had small groups of cells floating in the hemolymph and other distant sites, which suggests that tumor cells formed secondary multicellular growths (n>200). Dissection at various time points revealed that the majority of ectopic tumor cells progressively spread from the primary tumors onto the VNC, eventually enveloping it, and also spread into other tissues such as the first and second leg discs and tracheal vasculature at a lower frequency. All of the Ras^{V12}/scrib^{-/-} flies displayed the VNC invasion phenotype, making it the best measure of metastatic behavior. Confocal sections indicated that cells could invade the inside of the VNC, and the leading edge of these cells had a morphology and F-actin-rich periphery common to actively migrating cells. In transplantation experiments, the appearance of secondary growths varied considerably, regardless of genotype; however, only Ras^{V12}/scrib^{-/-} tumors invaded host tissues.

Ras^{V12}/scrib^{-/-} metastatic behavior was blocked with a UAS-scrib transgene, confirming the causal role of scrib inactivation (n=25). scrib inactivation causes the presence of overgrowths in homozygous flies (11). However, cells mutant for scrib alone grew poorly in the eye discs of either mosaic or zygotically mutant flies, and did not invade other tissues (n=100 and 10, respectively), which suggests that scrib inactivation and Ras^{V12} expression are both needed to cause metastatic behavior in eye disc cells.

We considered the possibility that excessive proliferation may cause a breakage of tissue boundaries, resulting in the appearance of ectopic GFP cells. However, other mutations identified in our screen caused excessive tumor growth without the appearance of ectopic cells. For example, clones of cells expressing Ras^{V12} and mutant for lats (Ras^{V12}/lats^{-/-}) resulted in immensely overgrown tumors in the eye-antennal discs (n=50). As shown by dissection at an early time point, Ras^{V12}/lats^{-/-} tumors grew faster than Ras^{V12}/scrib^{-/-} tumors, but ectopic cells were never seen on the VNC or other locations. Also, a weakly expressed eyFLP (eyFLP1.2) produced much smaller primary Ras^{V12}/scrib^{-/-} overgrowths than did eyFLP1, but these cells still exhibited metastatic behavior (n=10), indicating that excessive tumor growth alone does not cause metastatic behavior in Ras^{V12}/scrib^{-/-} larvae.

Basement membrane degradation contributes to tumor metastasis (12). To determine whether the basement membrane was compromised in Ras^{V12}/scrib^{-/-} tumors, we generated antibodies to laminin-A and used a GFP-tagged protein trap in the viking/collagen IV gene (13). When using the GFP-labeled collagen IV protein trap, mutant cells were marked with UAS-lacZ instead of UAS-GFP. The basement membrane was smooth and continuous on the outer surface of eye discs with wild-type, Ras^{V12}-expressing, or scrib^{-/-} cells. In discs containing Ras^{V12}/scrib^{-/-} cells, however, there were many points of discontinuity in the basement membrane, and mutant cells spread from these areas. Thus, like human malignant tumors, Drosophila metastatic tumors can acquire the ability to degrade basement membranes.

Mammalian invasive tumors commonly down-regulate E-cadherin through a variety of mechanisms, and this may be a causal factor in driving tumor progression (14). E-cadherin expression was lowered in Ras^{V12}/scrib^{-/-} cells, a phenotype likely due to inactivation of scrib. Expression of full-length E-cadherin (UAS-DECH) in Ras^{V12}/scrib^{-/-} cells suppressed their metastatic behavior (n=58). Expression of a truncated E-cadherin lacking its extracellular domain (UAS-CADH^{intra5}) no longer suppressed VNC invasion (n=15), indicating the importance of the cell adhesion function of E-cadherin in preventing metastatic behavior. We next examined whether down-regulation of shotgun (shg), the Drosophila E-cadherin homolog (15, 16), was sufficient to induce metastatic behavior in Ras^{V12}-expressing cells (Ras^{V12}/shg^{-/-}. Although Ras^{V12}/shg^{-/-} cells caused disc eversion defects during metamorphosis, mutant cells were not observed invading the VNC (n=21). This was confirmed with transplantation experiments, together suggesting that loss of E-cadherin is necessary but not sufficient for metastatic behavior in Ras^{V12}-expressing tumors.

scrib interacts with lethal giant larvae (lgl) and discs large (dlg) (11), and mutations in lgl or dlg when combined with Ras^{V12} expression caused metastatic behavior similar to that seen in Ras^{V12}/scrib^{-/-} flies (n=25 and 28, respectively). Aside from overgrowth phenotypes, mutations in scrib, lgl, and dlg also cause defects in cell polarity and epithelial monolayer formation (16). Other genes such as bazooka (baz), stardust (sdt), and cdc42 are also necessary for maintaining cell polarity, cell shape, and/or epithelial morphology, but their mutation does not result in overgrowth (17, 18). Inactivation of any one of these genes caused metastatic behavior when combined with Ras^{V12} expression (n=14, 10, and 10, respectively). Thus, alterations disrupting cell polarity and epithelial morphology play a key role in the development of metastatic behavior in noninvasive Ras^{V12}-expressing cells, perhaps through the abrogation of intercellular junctions or the mislocalization of plasma membrane-targeted signaling molecules. Interestingly, the identified genes function in an interdependent genetic hierarchy (19, 20), which suggests a concerted signaling pathway capable of suppressing metastatic behavior in tumor cells expressing Ras^{V12}.

Because oncogenic Ras promotes proliferation, survival, and cell growth in Drosophila (7, 21, 22), one or more of these processes may be sufficient to make scrib^{-/-} cells become metastatic. The p21 cyclin kinase inhibitor blocks Ras^{V12}-dependent proliferation in the Drosophila eye (9). Its expression in Ras^{V12}/scrib^{-/-} cells decreased tumor size but did not inhibit the ability of these cells to spread into the VNC (n=20). Expression of the oncogenic proliferation-stimulating proteins E2F and Dp (23) could not substitute for Ras^{V12} in scrib^{-/-} mutant cells (n=8). Expression of the p35 cell death inhibitor (24) also could not permit the metastatic progression of scrib^{-/-} mutant cells (n=10). Coexpression of E2F, Dp, and p35 visibly increased the amount of scrib^{-/-} mutant tissue, but again could not cause an induction of metastatic behavior (n=14). Expression of the growth-promoting oncogene homologs of c-Myc (25) or Akt (26) in scrib^{-/-} cells visibly increased the amount of mutant tissue, but neither of these could promote metastatic behavior (n=12 and 9, respectively). Inactivation of lats and scrib together (scrib^{-/-}, lats^{-/-}) resulted in tumors that did not exhibit metastatic behavior (n=16). Expression of dAkt in scrib^{-/-}, lats^{-/-} cells further increased tumor size, but again did not induce metastatic behavior (n=16). This indicates that increased proliferation, growth, and survival by means other than Ras^{V12} are not sufficient to cause the metastatic progression of scrib^{-/-} cells. Thus, the metastasis-promoting effect of scrib inactivation is highly dependent on its specific cooperation with the Ras^{V12} allele. Moreover, aside from its known effects on proliferation, growth, and survival, Ras^{V12} may function through an as yet undefined cellular mechanism to elicit metastatic progression in scrib^{-/-} cells.

Transplantation has been previously used to demonstrate that lethal giant larvae (lgl) and discs large (dlg) brains and wing discs can form secondary tumors (S1), implying that in Drosophila a single mutation may be sufficient to cause metastatic behavior. However, as tissues are cut into small pieces before transplantation, tumor formation could be aided by the disruption of basement membrane structure; interestingly, these mutant tissues have not been shown to exhibit invasiveness into adjacent tissues or to cause secondary tumor formation in situ. Also, as we have shown for scrib, clones of these mutant cells do not grown well in the presence of wild-type tissues (FIG. 2C), and not all discs in zygotically mutant animals are capable of overgrowth (FIG. 2D, S2, S3). The overgrowth phenotypes present in zygotically mutant scrib, lgl, and dlg larvae may be in part due to the disruption of signaling through nonautonomous factors such as Dpp, whose expression is perturbed in lgl mutants. This may explain why overgrowth in homozygous mutant animals is not recapitulated in mosaic clones.

Transplantation experiments were used to confirm whether the observed in situ behaviors of mutant cells could occur in a different environment. Transplanted tissues can be cultured in adult flies for several weeks, allowing ample time for tumors to invade host tissues. Transplantation of GFP-labeled RasV12/scrib−/− tumor fragments into the abdomens of wild-type adult females resulted in rapid tumor growth, caused a pronounced swelling of the abdomen, and quickly killed the host. Interestingly, this swelling was also seen in larvae with mosaic clones, suggesting that RasV12/scrib−/− cells could elicit an ascites-like host response. Control flies transplanted with tissue fragments only expressing RasV12 did not die as quickly, and never exhibited the swelling response to the transplant. Animals transplanted with disc fragments containing either wild-type or scrib loss of function cells did not die from the transplant, and the GFP-labeled tissue grew very slowly. Previous experiments have shown that some transplanted tumors can give rise to the appearance of secondary growths in distant locations such as the head. In our hands, we found that the appearance of such secondary growths was variable, which may be dependent upon the amount of primary tissue injected and the degree to which it or host tissues are disrupted during the process of transplantation. In contrast to these secondary focal points of growth, we detected a clear genotype-correlated invasive behavior when internal tissues from host animals were examined. Transplanted tissues with cells expressing RasV12, like those with wild-type or scrib loss-of-function cells, were present as distinct free-floating masses in the hemolymph. However, RasV12/scrib−/− tumors observed at the same time after transplantation were consistently attached to host tissues. Examination of host ovaries and intestines revealed GFP-positive cells both in developing ovarian follicles and the intestinal wall, suggesting that RasV12/scrib−/− cells migrated into and invaded these tissues. Such behavior was not observed for RasV12 or scrib single mutant cells. These data are consistent with the metastatic behavior observed in the genetically mosaic larvae carrying RasV12scrib−/− cells, indicating that the invasive phenotype was due to an intrinsic ability of these cells.

It has proven difficult to systematically study the genetic basis of metastasis with the currently available techniques. The Drosophila system described here circumvents the complication of acquired background mutations, which can occur through repeated passaging of cell lines or during the typically long latent period of mammalian tumor progression. In our initial screen, we found that mutations in different genes affecting the same physiological process—epithelial cell polarity maintenance—are sufficient in combination with Ras^{V12} to promote metastatic behavior in vivo. Interestingly, later stage human cancers typically lose cell polarity markers and epithelial structure during epithelial to mesenchymal transition (27). Also, E-cadherin loss, basement membrane degradation, and induction of cell migration and invasion relate well to observations made in human metastasis (12, 27, 28), which suggests that the ongoing screen will uncover genes and general mechanisms relevant to malignancy in humans.

It has been proposed that oncogenes such as Ras may play a dual role in tumorigenesis and metastasis (29); however, this has not yet been rigorously proven in mammalian systems, as the effects of Ras in cell culture depend greatly on the particular cell line used. We provide experimental evidence that genetic alterations promoting noninvasive tumor growth can indeed make additional contributions to the development of metastatic behavior, as Ras^{V12} expression is a crucial factor in making cell polarity-deficient cells metastasize. Furthermore, we show that oncogenic Ras specifically cooperates with inactivation of cell polarity genes to promote metastatic behavior. This may provide an explanation for the different metastatic potential observed in tumors of distinctive origins. The Drosophila genetics techniques described here should make it easier to analyze the specific targets of Ras^{V12} in metastatic cells, to identify other genes that cooperate with Ras^{V12} or other oncogenic alterations in promoting metastasis, and to elucidate the cellular processes that go awry during metastatic progression.

Materials and Methods

Strains

Flies were cultured at 25° C. on standard medium. The genotypes for the animals described in the text are listed below, along with the number of animals (n) dissected and analyzed for ventral nerve cord invasion.

For the F2 mosaic screens, w; UAS-RasV12; P[FRT]82B males were mutagenized and crossed with w; UAS-RasV12; Sb/TM6B females to obtain individual mutant lines balanced on the third chromosome. Alternatively, pre-existing mutations on 3R were recombined onto an FRT chromosome, and UAS-RasV12 was placed on the second chromosome. These lines were crossed with y,w,eyFLP1; Act5C>y+>Gal4, UASGFP/UAS-RasV12; P[FRT]82B Tub-Gal80 females and analyzed with a Leica MZ FLIII fluorescence stereomicroscope. UAS-scrib is an unpublished stock kindly given to us by David Bilder.
y,w,eyFLP1/w or Y; Act5C>y+>Gal4, UAS-GFP/+; P[FRT]82B Tub-Gal80/P[FRT]82B latsX1 (n>1000)
y,w,eyFLP1/w or Y; Act5C>y+>Gal4, UAS-GFP/+; P[FRT]82B Tub-Gal80/P[FRT]82B (n>1000)
y,w,eyFLP1/w or Y; Act5C>y+>Gal4, UAS-GFP/UAS-RasV12; P[FRT]82B Tub-Gal80/P[FRT]82B (n>1000)
y,w,eyFLP1/w or Y; Act5C>y+>Gal4, UAS-GFP/+; P[FRT]82B Tub-Gal80/P[FRT]82B, scrib1 (n=100)
y,w/w or Y; ey-FLP5, Act5C>y+>Gal4, UAS-GFP; scrib1/Df (3) Tl-X (n=10)
y,w,eyFLP1/w or Y; Act5C>y+>Gal4, UAS-GFP/UAS-RasV12; P[FRT]82B Tub-Gal80/P[FRT]82B, scrib1
orj7B3 (n>200)
y,w,eyFLP1/w, UAS-scrib; Act5C>y+>Gal4, UAS-GFP/UAS-RasV12; P[FRT]82B Tub-Gal80/P[FRT]82B, scrib1 (n=25)
y,w,eyFLP1/w or Y; Act5C>y+>Gal4, UAS-GFP/UAS-RasV12; P[FRT]82B Tub-Gal80/P[FRT]82B latsXTN33A or XI (n=50)
w,eyFLP1.2/w or Y; Act5C>y+>Gal4, UAS-GFP/UAS-RasV12; P[FRT]82B Tub-Gal80/P[FRT]82B, scrib1 (n=10)
y,w,eyFLP1/w or Y; Act5C>y+>Gal4, UAS-GFP/UAS-RasV12, UAS-DECH12; P[FRT]82B Tub-Gal80/P[FRT]82B scrib1 (n=58)
y,w,eyFLP1/w or Y; Act5C>y+>Gal4, UAS-GFP/UAS-RasV12, UAS-CADHintra5; P[FRT]82B Tub-Gal80/P[FRT]82B scrib1 (n=15)
y,w,eyFLP1/w or Y; P[FRT]43D Tub-Gal80/P[FRT]43D shg2 or k03401; Act5C>y+>Gal4, UAS-GFP/UAS-RasV12 (n=21)
y,w,eyFLP1/w or Y; lgl4 P[FRT]40A UAS-RasV12/Tub-Gal80 P[FRT]40A (n=25)
dlgm52 P[FRT]19A/Tub-Gal80 P[FRT]19A; eyFLP5, Act5C>y+>Gal4, UAS-GFP/UAS-RasV12 (n=28)
baz4 P[FRT]19A/Tub-Gal80 P[FRT]19A; eyFLP5, Act5C>y+>Gal4, UAS-GFP/UAS-RasV12 (n=14)
sdtN5 P[FRT]19A/Tub-Gal80 P[FRT]19A; eyFLP5, Act5C>y+>Gal4, UAS-GFP/UAS-RasV12 (n=10)
cdc421 P[FRT]19A/Tub-Gal80 P[FRT]19A; eyFLP5, Act5C>y+>Gal4, UAS-GFP/UAS-RasV12 (n=10)
y,w,eyFLP1/w or Y; Act5C>y+>Gal4, UAS-GFP/UAS-p21, UAS-RasV12; P[FRT]82B Tub-Gal80/P[FRT]82B, scrib1 (n=20)
y,w,eyFLP1/w or Y; Act5C>y+>Gal4, UAS-GFP/UAS-E2F, UAS-Dp; P[FRT]82B Tub-Gal80/P[FRT]82B scrib1 (n=8)
y,w,eyFLP1/w or Y; Act5C>y+>Gal4, UAS-GFP/UAS-p35; P[FRT]82B Tub-Gal80/P[FRT]82B scrib1 (n=10)
y,w,eyFLP1/w, UAS-p35; Act5C>y+>Gal4, UAS-GFP/UAS-E2F, UAS-Dp; P[FRT]82B Tub-Gal80/P[FRT]82B scrib1 (n=14)
y,w,eyFLP1/w or Y; Act5C>y+>Gal4, UAS-GFP/UAS-dMyc; P[FRT]82B Tub-Gal80/P[FRT]82B scrib1 (n=12)
y,w,eyFLP1/w or Y; Act5C>y+>Gal4, UAS-GFP/UAS-dAkt; P[FRT]82B Tub-Gal80/P[FRT]82B scrib1 (n=9)
y,w,eyFLP1/w or Y; Act5C>y+>Gal4, UAS-GFP/+; P[FRT]82B Tub-Gal80/P[FRT]82B scrib1, latsX1 (n=18)
y,w,eyFLP1/w or Y; Act5C>y+>Gal4, UAS-GFP/UAS-dAkt; P[FRT]82B Tub-Gal80/P[FRT]82B scrib1, latsX1 (n=16)
y,w,eyFLP1/w or Y; Act5C>y+>Gal4, UAS-lacZ/G454, UAS-RasV12; P[FRT]82B Tub-Gal80/P[FRT]82B
y,w,eyFLP1/w or Y; Act5C>y+>Gal4, UAS-lacZ/G454, UAS-RasV12; P[FRT]82B Tub-Gal80/P[FRT]82B, scrib1
   Analsis of Tissues

Cephalic complexes of wandering third instar larvae were dissected and the pattern of GFP expression in mutant clones was carefully observed in eye/antennal discs, the brain, and the leg discs. The presence of GFP-expressing cells in other areas of the larvae was also noted with the exception of cells in the gonads and genital discs (as well as the leg discs for eyFLP5 and wing discs for eyFLP1.2), as this was also observed in animals with wild-type and otherwise noninvasive clones. Invasive tumors were considered suppressed if the majority of animals could pupate, and the majority of dissected cephalic complexes had little (minor projections restricted to the anterior of the VNC) to no GFP-expressing cells invading the VNC.

Transplatation

Transplantation of imaginal disc tissues into adult hosts was performed as previously described (S1), with the exception that flies were anesthetized with CO2 rather than ether. Briefly, third instar eye imaginal discs were dissected in PBS, cut into small pieces, and injected into the abdomens of at least 2-day-old well-fed females. Individual females were placed in a vial with several males, and any females that died within the first day were discarded and not included in the mortality curve. Pictures of GFP-marked tissue were taken every other day, and GFP expression away from the primary tumor was recorded. For the mortality curve, 15 animals of each genotype were analyzed. For internal organ analysis, 3 animals of each genotype were dissected on day 6 or 12 after transplant.

Immunohistochemistry

Double and triple immunofluorescent labeling of third instar imaginal discs was performed according to standard protocol and was visualized using FITC-, CY5- or CY3-conjugated secondary antibodies (Jackson Labs). Tissues were mounted in a manner whereby cross-sections of epithelial layers could be visualized, and all analyses were performed on undifferentiated cells in the eye-antennal disc anterior to the morphogenetic furrow. a-DCAD1 (rat monoclonal antibody against Drosophila E-cadherin) was from T. Uemura (1:20). Rhodamine-conjugated phalloidin was from Sigma (1:100). abGalactosidase (1:400) was from ICN Biomed. a-Laminin rabbit antiserum (1:100) was generated with a combination of the synthesized Laminin A peptides

RKIYATATCGPDTDGPELYCKGGGC,
CGGGMINITPNMVVGDIWQGYCPLN,
and
CGGGKYIVAPDVILFSEHNALVHTS.

1. A. F. Chambers, A. C. Groom, I. C. MacDonald, Nature Rev. Cancer 2, 563 (2002).
2. P. S. Steeg, Nature Rev. Cancer 3, 55 (2003).
3. C. J. Potter, G. S. Turenchalk, T. Xu, Trends Genet. 16, 33 (2000).
4. R. A. Pagliarini, A. T. Quinones, T. Xu, in Tumor Suppressor Genes: Regulation, Function, and Medicinal Applications, W. S. El-Deiry, Ed. (Humana, Totowa, N.J., 2003), pp. 349-381.
5. T. P. Newsome, B. Asling, B. J. Dickson, Development 127, 851 (2000).
6. M. A. St. John et al., Nature Genet. 21, 182 (1999).
7. F. D. Karim, G. M. Rubin, Development 125, 1 (1998).
8. G. H. Fisher et al., Genes Dev. 15, 3249 (2001).
9. T. Xu, W. Wang, S. Zhang, R. A. Stewart, W. Yu, Development 121, 1053 (1995).
10. D. Bilder, N. Perrimon, Nature 403, 676 (2000).
11. D. Bilder, M. Li, N. Perrimon, Science 289, 113 (2000).
12. M. Egeblad, Z. Werb, Nature Rev. Cancer 2, 161 (2002).
13. X. Morin, R. Daneman, M. Zavortink, W. Chia, Proc. Natl. Acad. Sci. U.S.A. 98, 15050 (2001).
14. A. K. Perl, P. Wilgenbus, U. Dahl, H. Semb, G. Christofori, Nature 392, 190 (1998).
15. T. Uemura et al., Genes Dev. 10, 659 (1996).
16. U. Tepass et al., Genes Dev. 10, 672 (1996).
17. J. L. Genova, S. Jong, J. T. Camp, R. G. Fehon, Dev. Biol. 221, 181 (2000).
18. H. A. Muller, E. Wieschaus, J. Cell Biol. 134, 149 (1996).
19. D. Bilder, M. Schober, N. Perrimon, Nature Cell Biol. 5, 53 (2003).
20. G. Tanentzapf, U. Tepass, Nature Cell Biol. 5, 46 (2003).
21. A. Bergmann, J. Agapite, K. McCall, H. Steller, Cell 95, 331 (1998).
22. D. A. Prober, B. A. Edgar, Cell 100, 435 (2000).
23. W. Du, J. E. Xie, N. Dyson, EMBO J. 15, 3684 (1996).
24. B. A. Hay, T. Wolff, G. M. Rubin, Development 120, 2121 (1994).
25. L. A. Johnston, D. A. Prober, B. A. Edgar, R. N. Eisenman, P. Gallant, Cell 98, 779 (1999).
26. J. Verdu, M. A. Buratovich, E. L. Wilder, M. J. Birnbaum, Nature Cell Biol. 1, 500 (1999).
27. J. P. Thiery, Nature Rev. Cancer 2, 442 (2002).
28. P. Guilford, Mol. Med. Today 5, 172 (1999).
29. R. Bernards, R. A. Weinberg, Nature 418, 823 (2002).
30. T. Xu, G. M. Rubin, Development 117, 1223 (1993).
31. T. Lee, L. Luo, Neuron 22, 451 (1999).
32. K. Ito, W. Awano, K. Suzuki, Y. Hiromi, D. Yamamoto, Development 124, 761 (1997).
33. A. H. Brand, N. Perrimon, Development 118, 401 (1993).
34. M. Therrien, A. M. Wong, G. M. Rubin, Cell 95, 343 (1998).

### EXAMPLE 2

### Loss of Cell Polarity Drives Tumor Growth and Metastasis through JNK Activation in Drosophila

In Example 1, we described a genetic screen in Drosophila to identify genes that, when homozygously mutated, will promote aspects of tumor progression and metastasis in otherwise benign tumors generated through expression of oncogenic Ras, a common occurrence in human cancers (Malumbres and Barbacid, 2003; Pagliarini and Xu, 2003). Initial screening indicated that loss of Drosophila genes involved in cell polarity maintenance, such as scribble (scrib), lethal giant larvae (lgl), and discs large (dlg) can cooperate with oncogenic Ras to promote excess tumor growth, basement membrane degradation, loss of E-cadherin, local invasion, and formation of secondary tumor foci. Scrib, Lgl, and Dlg are evolutionarily conserved proteins that function at the lateral domains of epithelial cell membranes to maintain apicobasal polarity (Bilder, 2004; Humbert et al., 2003; Macara, 2004). Each protein requires the others for proper localization and/or stability, and genetic studies have shown that they function in a common genetic pathway (Bilder et al., 2000). Indeed, disruption of any one of the genes encoding these proteins produces similar apicobasal polarity defects including failure of adherens junction assembly as well as the spreading of apical markers toward the basal surface. In addition to a role in cell polarity, these three proteins are also involved in regulation of cell proliferation. Zygotic loss of scrib, lgl or dlg results in the neoplastic overgrowth of larval imaginal discs and the brain lobes, leading to the classification of these three genes as Drosophila neoplastic tumor suppressors (Bilder, 2004; Gateff, 1994; Wodarz, 2000). Since the loss of cell polarity is a crucial step for tumor malignancy, it is quite important to elucidate the poorly understood molecular mechanisms that dictate how polarity and proliferation control can be coupled in these mutant cells.

Interestingly, neoplastic overgrowth can not be seen if the cell polarity gene mutations are present as clones of cells in otherwise wild-type tissues (Arquier et al., 2001; Brumby and Richardson, 2003; Pagliarini and Xu, 2003). These mutant cells are apparently outcompeted by the growth of wild type cells in developing eye discs. A fuller understanding of how these gene mutations can cooperate with oncogenic Ras to promote tumor growth and metastatic behavior requires a deeper understanding of the signaling pathways perturbed in scrib, lgl, and dlg mutant animals. It has been shown that loss of scrib^{-/-} mutant cells during development can be inhibited by blocking c-Jun N-terminal kinase (JNK) signaling, suggesting that cell polarity-deficient cells might be eliminated by cell death caused by JNK activation (Brumby and Richardson, 2003). This cell death might be inhibited through overexpression of oncogenic Ras (Ras^{V12}), as the combination of these two resulted in enhanced tumor growth, although this has not been rigorously proven (Brumby and Richardson, 2003; Pagliarini and Xu, 2003); in addition, it has not been shown whether these putative perturbations in JNK activity can also play an oncogenic role, as they do in some human cancers (Davis, 2000; Ip and Davis, 1998).

In this study, we show that a crucial oncogenic effect of cell polarity gene inactivation is the activation of JNK signaling. Genetic evidence in Drosophila reveals that JNK activation is essential for both the accelerated tumor growth and metastatic behavior of tumors induced by a combination of Ras^{V12} and loss of cell polarity gene function. We show evidence that cell polarity-deficient cells are eliminated by INK-mediated cell death, and this cell death is blocked by oncogenic Ras^{V12}, resulting in constitutive activation of INK signaling. As a result, simultaneous activation of JNK and Ras pathways results in a massive tumor growth. Furthermore, we combined activation of the JNK and Ras pathways with disruption of the E-cadherin/catenin epithelial cell adhesion complex through a loss-of-function mutation in the β-catenin gene. These three genetic alterations in conserved cancer signaling pathways reconstitute the metastatic phenotype in Drosophila. Our findings demonstrate that activation of JNK signaling is crucial in promoting growth and metastasis of Drosophila tumors initiated through oncogenic Ras.

Results

In analyzing the global expression profile of Drosophila eye disc tumors generated through expression of oncogenic Ras^{V12} and loss of scrib (hereafter referred to as Ras^{V12}/scrib^{-/-}, Pagliarini and Xu, 2003), we observed that expression of the JNK phosphatase puckered (puc) was strongly up-regulated in metastatic tumors. The up-regulation of puc indicates activation of the JNK pathway in Drosophila (Adachi-Yamada et al., 1999a). JNK was originally identified as a stress-activated protein kinase that phosphorylates the c-Jun oncoprotein, and its role in stress-induced cell death has been well documented (Davis, 2000). In recent years, JNK has emerged as a crucial regulator of a variety of biological processes including epithelial proliferation and migration (Davis, 2000; Kockel et al., 2001; Xia and Karin, 2004; Zhang et al., 2004). JNK has also shown to be essential for migration of cultured tumor epithelial cells (Huang et al., 2003). In addition, JNK has been implicated in tumor development through its functional interaction with oncogene products (Davis, 2000; Ip and Davis, 1998). These facts prompted us to pursue JNK as an attractive candidate for orchestrating enhanced tumor growth and metastasis in Ras^{V12}/scrib^{-/-} tumors.

Ras^{V12}/scrib^{-/-}, Ras^{V12}/lgl^{-/-}, or Ras^{V12}/dlg^{-/-} mutant clones induced in developing larval eye discs all generate tumors with identical metastatic behavior, while clones of cells expressing Ras^{V12} do not invade (Pagliarini and Xu, 2003). In addition, genetic studies have shown that Scrib, Lgl, and Dlg act in a common pathway as a cassette, regulating a single process to organize cell polarity (Bilder et al., 2000). We therefore utilized Ras^{V12}/scrib^{-/-}, Ras^{V12}/lgl^{-/-}, and Ras^{V12}/dlg^{-/-} clones as interchangeable metastatic tumor models, as it would have been impossible to recombine all previously published transgenes used in this study into the same parent fly strain. However, transgenes and mutations were tested in multiple parent strains when possible. Green fluorescent protein (GFP)-labeled noninvasive Ras^{V12} tumors gradually grew in a time-dependent manner (FIGS. 1A-D and 2A-D). A small minority of Ras^{V12}-expressing animals lived beyond day 6 (FIGS. 1D and 2D). On the other hand, metastatic Ras^{V12}/lgl^{-/-} tumors dramatically grew during 5-6 days after egg lay, and continued to grow up to 15 days. The metastatic tumors outcompeted surrounding wild-type cells, resulting in a loss of the unlabeled wild type cells and a dramatic increase in the GFP-expressing mutant tissue.

To examine the possible role of JNK signaling in this system, we introduced an enhancer-trap allele, puc-LacZ, to monitor JNK activity (Adachi-Yamada et al., 1999a; Martin-Blanco et al., 1998). Strong ectopic JNK activation was seen in the eye discs with metastatic tumors at day 4 and later while noninvasive tumors only showed JNK activation in the anterior margin and posterior parts of eye-antennal discs at late larval stages, where JNK is normally activated in wild-type animals (Agnes et al., 1999; Igaki et al., 2002). Confocal images revealed that JNK signaling was activated within the metastatic tumor clones. Intriguingly, intense staining was seen in the cells extending projections and invading the brain hemispheres and ventral nerve cord. Some metastasizing cells were observed even at day 4, the time point before these cells acquire a dramatic growth advantage, indicating that tumor overgrowth is not required for acquiring the metastatic phenotype. The ectopic activation of JNK in Drosophila metastatic tumor cells, particularly in the actively invading cells, suggests it is playing a functional role in tumor progression. To assess this possibility, we blocked JNK signaling in metastatic tumors by overexpressing a dominant-negative (DN) form of Basket, the Drosophila JNK (Bsk^{DN}, Adachi-Yamada et al., 1999b). Inhibition of JNK activation strongly suppressed the massive tumor growth induced by Ras^{V12}/scrib^{-/-}, Ras^{V12}/lgl^{-/-} or Ras^{V12}/dlg^{-/-}. In addition, the metastatic migration of these cells toward the brain region was completely blocked by Bsk^{DN}. Similar results were also obtained when Bsk^{DN} was overexpressed in metastatic tumors induced by other cell polarity mutations, bazooka^{-/-}, stardust^{-/-}, or cdc42^{-/-} (Pagliarini and Xu, 2003) in conjunction with Ras^{V12} expression. Together, this shows that signaling through the JNK cascade is necessary to mediate tumor progression and metastatic behavior in Ras^{V12} cells also mutant for cell polarity genes.

To further dissect the signaling molecules involved in this phenomenon, we tested genetic interactions with other JNK pathway components. The JNK pathway consists of sequential activation of JNK kinase kinase (JNKKK), JNK kinase (JNKK), and JNK, then transduces the signal through phosphorylation of target proteins such as c-Jun (Davis, 2000; Ip and Davis, 1998). Some of the best characterized upstream molecules that initiate this kinase cascade are members of the Tumor necrosis factor Receptor Associating Factor (TRAF) family of proteins, the adaptor proteins that mediate signals from cell surface receptors to cytoplasmic effectors (Bradley and Pober, 2001; Varfolomeev and Ashkenazi, 2004). We observed that both the enhanced overgrowth and metastatic behavior in Ras^{V12}/dlg^{-/-}, Ras^{V12/}lgl^{-/-}, or Ras^{V12}/scrib^{-/-} tumors were strongly suppressed by RNAi-mediated “knock-down” of Drosophila TRAF2 (DTRAF2), a fly homolog of TRAF6, or by knock-down of dTAK1, a Drosophila JNKKK, but not by RNAi of DTRAF1. Involvement of dTAK1 in this signaling cascade was confirmed by overexpression of dTAK1^{DN} (Mihaly et al., 2001). All genotypes displayed 100% phenotypic penetrance except for two genotypes with RNAi constructs, DTRAF2-IR and dTAK1-IR, which still showed 89% (17/19) and 63% (26/41) penetrance, respectively. Together, these observations reveal that activation of JNK signaling specifically through DTRAF2 and dTAK1 is essential for both enhanced tumor growth and metastasis in this system.

JNK mediates cell death signaling in both mammals and Drosophila (Davis, 2000). A recent study has shown that the size of scrib^{-/-} mutant clones generated in the eye disc was reduced relative to wild type cells during development and that expression of Bsk^{DN} within these cells suppressed this size reduction (Brumby and Richardson, 2003). This suggests that cells with apicobasal polarity defects might be eliminated by cell death through JNK activation. Since cell death should act as a negative regulator for tumor growth, it could be compromised by Ras^{V12}-induced survival signaling in these tissues. To test this possibility, we generated dlg^{-/-} clones in developing eye discs and assessed JNK activity and cell death. Strong puc-LacZ expression was seen within dlg^{-/-} clones, indicating cell polarity disruption is sufficient to cause JNK activation. Acridine orange staining of these tissues revealed that dlg^{-/-} cells were indeed dying. Interestingly, cell death was completely abolished in Ras^{V12}/dlg^{-/-} clones despite showing strong JNK activation. This blockage of cell death by Ras^{V12} was also observed in scrib^{-/-} clones. In addition, cell death in scrib^{-/-} clones was blocked by expression of Bsk^{DN}, confirming that cell death in these mutants indeed occurs through JNK activation. These results suggest that activation of Ras signaling specifically blocks the JNK-mediated cell death signal induced by cell polarity disruption.

The observations that JNK activation was required for enhanced overgrowth of metastatic tumors but that JNK activation by itself resulted in cell death raised the possibility that Ras signaling and JNK signaling cooperate with each other to promote tumor growth. To test this idea, we overexpressed the Drosophila tumor necrosis factor (TNF) ligand Eiger, an activator of JNK signaling (Igaki et al., 2002; Moreno et al., 2002b), within Ras^{V12}-expressing clones. Intriguingly, accelerated tumor growth was observed in Ras^{V12}/Eiger-expressing clones at day 10-14, although neither Ras^{V12} alone nor Eiger alone caused dramatic overgrowth or a dramatically extended larval period. This massive overgrowth was completely blocked by co-expression of Bsk^{DN}, demonstrating this phenotype was caused by cooperation between Ras signaling and JNK signaling.

Tumor invasion of the brain hemispheres and VNC was completely blocked by inhibition of JNK signaling, suggesting a possible role of JNK signaling in tumor cell invasion as well as in enhanced tumor growth. However, coexpression of Ras^{V12} and Eiger did not reconstitute the invasive phenotype, indicating that some other events are required for establishing metastasis. A strong candidate for the missing event is down-regulation of E-cadherin signaling, since we have previously found that a loss of E-cadherin is a necessary effect of scrib loss in promoting the metastatic behavior of Ras^{V12}/scrib^{-/-} tumors (Pagliarini and Xu, 2003). E-cadherin-mediated cell-cell adhesion requires a complex series of interactions between E-cadherin and catenins in the cytoplasm to link E-cadherin to the actin cytoskeleton (Cavallaro and Christofori, 2004). The E-cadherin/catenin adhesion complex is frequently down-regulated in malignant tumor cells, and the inactivation of this complex could be a prerequisite for tumor progression and metastasis (Bremnes et al., 2002; Cavallaro and Christofori, 2004; Guilford et al., 1998; Hirohashi, 1998; Wijnhoven et al., 2000). In addition, genetic alterations in β-catenin abolishing cell-cell adhesiveness have been observed in two gastric cancer cell lines (Kawanishi et al., 1995; Oyama et al., 1994). As with E-cadherin, Drosophila β-catenin, also known as armadillo (arm), was down-regulated and its polarized apical localization was disrupted within Ras/scrib^{-/-} clones. This knowledge allowed us to test how disruption of cell adhesion interplayed with metastasis through loss of β-catenin in Ras^{V12}-induced tumors. Loss-of-function mutations in arm in conjunction with Ras^{V12} expression resulted in a disorganized eye disc but did not show any metastatic behavior, similarly to loss of E-cadherin in Ras^{V12} tumors (Pagliarini and Xu, 2003). However, coexpression of Ras^{V12} and Eiger combined with a loss of arm induced invasion of the VNC and slightly increased growth of tumors in the eye discs. This indicates that oncogenic Ras, JNK activation, and a loss of E-cadherin/catenin complex function are the minimal components to induce metastatic tumors in Drosophila eye discs. In addition, these results suggest that JNK and Ras^{V12} cooperate with each other to promote metastasis independently of their ability to promote tumor growth.

Discussion

Mammalian epithelial tumor cells lose their polarity during tumor progression toward malignancy; however, the mechanistic aspect how cell polarity disruption contributes to this process remain, for the most part, undiscovered. In Drosophila, loss-of-function mutations in scrib, lgl or dlg result in a disruption of cell polarity in epithelia and neuroblasts, and simultaneously induce neoplastic overgrowth of these cells. The fly neoplastic tumors share several features with human malignant tumors including overproliferation, loss of epithelial architecture, failure to differentiate, and invasive characteristics (Bilder, 2004). These facts indicate that it is clearly important to elucidate the basic principles by which cell polarity and proliferation control are linked, and that genetic analysis in Drosophila provides a powerful system in which to study this issue.

We previously showed that Ras^{V12} tumors acquire enhanced growth and metastatic behavior with a loss of cell polarity, but the mechanisms of how cell polarity disruptions promote these biological outcomes has not been understood. Our previous results showing that loss of E-cadherin was necessary but not sufficient for metastatic behavior suggested that an additional pathway was important for the dramatic cooperation between Ras^{V12} and cell polarity defects. In this report, we show that cell polarity disruption potently activates JNK signaling, which promotes the massive growth and metastatic behavior of Ras^{V12} tumors. Although Ras^{V12} alone only induces moderate tumor growth (FIG. 2A), and loss of cell polarity alone causes JNK-mediated cell death (FIG. 2B), cooperation between these two pathways promotes tumor progression in three ways: 1) combined activation of JNK and Ras pathways causes enhanced tumor overgrowth, 2) JNK-induced cell death signaling is blocked by Ras^{V12}, and 3) both JNK signaling and Ras signaling are essential for establishing metastatic invasiveness (FIG. 2C). Our findings therefore indicate that the pro-apoptotic function of JNK in cell polarity-deficient cells can be converted to a pro-tumor effect in the presence of oncogenic Ras.

The JNK signaling is essential for a variety of biological processes such as morphogenesis, inflammation, cell proliferation, cell migration, planar cell polarity, and cell death (Davis, 2000; Ip and Davis, 1998). Genetic studies in Drosophila have demonstrated that JNK signaling is essential for epithelial cell movements (Ip and Davis, 1998; Kockel et al., 2001). In addition, a genetic study in mice revealed that TNF receptor-JNK signaling stimulates epidermal proliferation (Zhang et al., 2004). Moreover, JNK is constitutively activated in several tumor cell lines, and the transforming activity of several oncogenes are JNK dependent (Ip and Davis, 1998). These studies suggest that JNK may play an important role in tumorigenesis, tumor growth, and metastasis. Interestingly, Ras-induced transformation requires c-Jun (Johnson et al., 1996), and Ras induces phosphorylation of c-Jun on sites also phosphorylated by JNK (Pulverer et al., 1991; Smeal et al., 1991). Furthermore, in Drosophila, Ras and JNK signaling both phosphorylate the Drosophila c-Fos homolog; each pathway phosphorylates different residues, and the biological outcome is dependent upon the additive effects of Ras and JNK signaling (Ciapponi et al., 2001). These studies suggest a possible collaboration between Ras and JNK in tumor development. In this report, we provide the first genetic evidence that oncogenic Ras and JNK cooperate with each other to promote both tumor growth and metastasis. A possible mechanism for triggering the JNK activation by cell polarity disruption is through a perturbation in ligand/receptor-mediated signals. Cell polarity defects may cause a mislocalization of cell surface receptors or ligands, which are normally tightly regulated in polarized epithelial cells, resulting in stimulation or attenuation of their signals. JNK signaling might be directly activated by a ligand/receptor signaling such as Eiger/Wengen signaling (Igaki et al., 2002; Kanda et al., 2002), or might be indirectly activated through a down-regulation of survival signaling such as reduced Dpp signaling (Moreno et al., 2002a). Involvement of DTRAF2 in this signaling supports this idea, since TRAF proteins bind to intracellular portion of cell surface receptors to mediate their signals (Bradley and Pober, 2001). Alternatively, the cell polarity defect may directly affect the activity of DTRAF2 by influencing its polyubiquitination or by regulating its putative negative regulators such as IRAK-M in mammals (Wu and Arron, 2003).

In this study, we have shown that JNK activation and cell adhesion complex loss are sufficient to account for the loss of the cell polarity genes (FIG. 2C). The reconstituted metastatic tumors, however, showed relatively smaller tumors and fewer metastatic cells compared with that caused by the combination of Ras^{V12} and cell polarity disruption. This difference could be due to a lack of the potential neoplastic growth ability provided by cell polarity disruption (Bilder, 2004). In addition, tumor cell viability might be reduced by a blockage of Wingless signaling, a survival signal in which arm is involved as a signal transducer (Giraldez and Cohen, 2003). Further elucidation of how signaling pathways are perturbed in scrib, lgl, and dlg mutant will no doubt extend our mechanistic knowledge of how the evolutionarily conserved JNK and cadherin/catenin signaling mediate metastasis.

The Ras oncogene is activated in about 30% of human cancers (Hanahan and Weinberg, 2000). Although Ras has been implicated in metastatic processes in mammalian systems, the mechanistic aspect of Ras' function in metastasis is largely unknown (Bernards and Weinberg, 2002). In our metastasis model system, other growth promoting alterations can not substitute for Ras^{V12} in scrib^{-/-} cells to cause enhanced tumor growth and metastasis (Brumby and Richardson, 2003; Pagliarini and Xu, 2003), indicating that a specific aspect of Ras signaling is required for inducing these phenotypes. Further genetic analysis using the fly model system may provide novel ideas for understanding tumor progression and metastasis in humans through the elucidation of specific downstream targets of Ras and JNK cooperative activity. In addition, it will be interesting to confirm if inhibition of JNK signaling in mammalian tumors bearing oncogenic Ras is a valid therapeutic target for preventing metastasis. Since the JNK pathway is a kinase cascade, it is potentially amenable to small molecule inhibition (Huang et al., 2003). Further studies combining large-scale gene identification and signaling pathway analysis in Drosophila with gene validation through retrospective analysis of mutations in human cancer tissues will lead to a development of a novel therapeutic strategy against cancers.

Methods

Generation of Marked Clones

Fluorescently-labeled noninvasive or metastatic tumors (using either UAS-GFP or UAS-RFP) were produced in the eye disc as previously described (Pagliarini and Xu, 2003). Briefly, eyeless promoter-driven FLP recombinase (eyFLP) (Newsome et al., 2000) was used to activate Act>y+>Gal4 ‘flip-out’ construct-driven UAS-transgene expression (Ito et al., 1997). This allowed expression of UAS-GFP (or UAS-RFP) to positively mark mutant tissue, as well as expression of UAS-Ras^{V12} and other transgenes to functionally analyze tumor biology. eyFLP also promoted FRT-mediated mitotic recombination, which allowed for both removal of the Tub-Gal80 repressor (MARCM system, (Lee and Luo, 1999) and loss of heterozygosity of Drosophila tumor suppressor genes (Xu et al., 1995). Depending on the chromosomal location of loss-of-function mutations to be analyzed, mitotic recombination was induced on chromosome X, 2L, or 3R using FRT insertions at cytological locations 19A, 40A, or 82B, respectively.

Histology

Larval tissues were immunostained using standard procedures for confocal microscopy. A rabbit anti-β-galactosidase antibody (Cappel, 1: 200) and a Cy3-conjugated secondary antibody (Jackson Labs) were used to monitor JNK activation in the genetic background of puc-LacZ (Adachi-Yamada et al., 1999a; Martin-Blanco et al., 1998). N2 7A1 (mouse α-armadillo antibody, Developmental Studies Hybridoma Bank, 1:20) and a Cy5-conjugated secondary antibody (Jackson Labs) were used for anti-armadillo staining. Images were acquired using a Zeiss Axioplan 2 microscope with a BioRad 1240 Confocal microscope. For the analysis of metastatic behavior, the pattern of GFP-expressing mutant cells was carefully observed in the eye discs, brain hemispheres, and VNC using a Leica MZ FLIII fluorescence stereomicroscope and confocal microscope described above.

Fly Strains

The following strains were used: UAS-Ras^{V12} (Karim and Rubin, 1998), Tub-Gal80, FRT19A; eyFLP5, Act>y⁺>Gal4, UAS-GFP (“19A tester”) (Pagliarini and Xu, 2003), yw,eyFLP1; Tub-Gal80, FRT40A; Act>y⁺>Gal4, UAS-GFP (“40A tester”) (Pagliarini and Xu, 2003), yw,eyFLP1; Act>y⁺>Gal4, UAS-GFP; FRT82B, Tub-Gal80 (“82B tester”) (Pagliarini and Xu, 2003), UAS-Eiger (Igaki et al., 2002), UAS-Bsk^{DN} (Adachi-Yamada et al., 1999b), UAS-DTRAF1-IR and UAS-DTRAF2-IR (Xue et al., manuscript in preparation; M. Miura, personal communication), UAS-dTAK1-IR (Leulier et al., 2002), UAS-dTAK1^{K46R} (Mihaly et al., 2001), scrib¹ (Bilder and Perrimon, 2000), lgl⁴ (Gateff and Schneiderman, 1967), dlg^{m52} (Goode and Perrimon, 1997), puc^{E69} (Martin-Blanco et al., 1998), and arm¹ (Peifer et al., 1991). UAS-srcRFP was a kind gift of H. C. Chang.

### REFERENCES FOR EXAMPLE 2

Adachi-Yamada, T., Fujimura-Kamada, K., Nishida, Y., and Matsumoto, K. (1999a). Distortion of proximodistal information causes JNK-dependent apoptosis in Drosophila wing. Nature 400, 166-169.

Adachi-Yamada, T., Gotoh, T., Sugimura, I., Tateno, M., Nishida, Y., Onuki, T., and Date, H. (1999b). De novo synthesis of sphingolipids is required for cell survival by down-regulating c-Jun N-terminal kinase in Drosophila imaginal discs. Mol Cell Biol 19, 7276-7286.

Agnes, F., Suzanne, M., and Noselli, S. (1999). The Drosophila JNK pathway controls the morphogenesis of imaginal discs during metamorphosis. Development 126, 5453-5462.

Arquier, N., Perrin, L., Manfruelli, P., and Semeriva, M. (2001). The Drosophila tumor suppressor gene lethal(2)giant larvae is required for the emission of the Decapentaplegic signal. Development 128, 2209-2220.

Bernards, R., and Weinberg, R. A. (2002). A progression puzzle. Nature 418, 823.

Bilder, D. (2004). Epithelial polarity and proliferation control: links from the Drosophila neoplastic tumor suppressors. Genes Dev 18, 1909-1925.

Bilder, D., Li, M., and Perrimon, N. (2000). Cooperative regulation of cell polarity and growth by Drosophila tumor suppressors. Science 289, 113-116.

Bilder, D., and Perrimon, N. (2000). Localization of apical epithelial determinants by the basolateral PDZ protein Scribble. Nature 403, 676-680.

Bradley, J. R., and Pober, J. S. (2001). Tumor necrosis factor receptor-associated factors (TRAFs). Oncogene 20, 6482-6491.

Bremnes, R. M., Veve, R., Hirsch, F. R., and Franklin, W. A. (2002). The E-cadherin cell-cell adhesion complex and lung cancer invasion, metastasis, and prognosis. Lung Cancer 36, 115-124.

Brumby, A. M., and Richardson, H. E. (2003). scribble mutants cooperate with oncogenic Ras or Notch to cause neoplastic overgrowth in Drosophila. Embo J 22, 5769-5779.

Cavallaro, U., and Christofori, G. (2004). Cell adhesion and signalling by cadherins and Ig-CAMs in cancer. Nat Rev Cancer 4, 118-132.

Chambers, A. F., Groom, A. C., and MacDonald, I. C. (2002). Dissemination and growth of cancer cells in metastatic sites. Nat Rev Cancer 2, 563-572.

Ciapponi, L., Jackson, D. B., Mlodzik, M., and Bohmann, D. (2001). Drosophila Fos mediates ERK and JNK signals via distinct phosphorylation sites. Genes Dev 15, 1540-1553.

Davis, R. J. (2000). Signal transduction by the JNK group of MAP kinases. Cell 103, 239-252.

Gateff, E. (1994). Tumor suppressor and overgrowth suppressor genes of Drosophila melanogaster: developmental aspects. Int J Dev Biol 38, 565-590.

Gateff, E. and Schneiderman, H. A. (1967). Developmental studies of a new mutant of Drosophila melanogaster: Lethal malignant brain tumor (1(2)gl 4). Am Zool 7, 760

Giraldez, A. J., and Cohen, S. M. (2003). Wingless and Notch signaling provide cell survival cues and control cell proliferation during wing development. Development 130, 6533-6543.

Goode, S., and Perrimon, N. (1997). Inhibition of patterned cell shape change and cell invasion by Discs large during Drosophila oogenesis. Genes Dev 11, 2532-2544.

Guilford, P., Hopkins, J., Harraway, J., McLeod, M., McLeod, N., Harawira, P., Taite, H., Scoular, R., Miller, A., and Reeve, A. E. (1998). E-cadherin germline mutations in familial gastric cancer. Nature 392, 402-405.

Hanahan, D., and Weinberg, R. A. (2000). The hallmarks of cancer. Cell 100, 57-70.

Hingorani, S. R., Petricoin, E. F., Maitra, A., Rajapakse, V., King, C., Jacobetz, M. A., Ross, S., Conrads, T. P., Veenstra, T. D., Hitt, B. A., et al. (2003). Preinvasive and invasive ductal pancreatic cancer and its early detection in the mouse. Cancer Cell 4, 437-450.

Hirohashi, S. (1998). Inactivation of the E-cadherin-mediated cell adhesion system in human cancers. Am J Pathol 153, 333-339.

Huang, C., Rajfur, Z., Borchers, C., Schaller, M. D., and Jacobson, K. (2003). JNK phosphorylates paxillin and regulates cell migration. Nature 424, 219-223.

Humbert, P., Russell, S., and Richardson, H. (2003). Dlg, Scribble and Lgl in cell polarity, cell proliferation and cancer. Bioessays 25, 542-553.

Igaki, T., Kanda, H., Yamamoto-Goto, Y., Kanuka, H., Kuranaga, E., Aigaki, T., and Miura, M. (2002). Eiger, a TNF superfamily ligand that triggers the Drosophila JNK pathway. Embo J 21, 3009-3018.

Ip, Y. T., and Davis, R. J. (1998). Signal transduction by the c-Jun N-terminal kinase (JNK)—from inflammation to development. Curr Opin Cell Biol 10, 205-219.

Ito, K., Awano, W., Suzuki, K., Hiromi, Y., and Yamamoto, D. (1997). The Drosophila mushroom body is a quadruple structure of clonal units each of which contains a virtually identical set of neurones and glial cells. Development 124, 761-771.

Johnson, R., Spiegelman, B., Hanahan, D., and Wisdom, R. (1996). Cellular transformation and malignancy induced by ras require c-jun. Mol Cell Biol 16, 4504-4511.

Kanda, H., Igaki, T., Kanuka, H., Yagi, T., and Miura, M. (2002). Wengen, a member of the Drosophila tumor necrosis factor receptor superfamily, is required for Eiger signaling. J Biol Chem 277, 28372-28375.

Karim, F. D., and Rubin, G. M. (1998). Ectopic expression of activated Ras1 induces hyperplastic growth and increased cell death in Drosophila imaginal tissues. Development 125, 1-9.

Kawanishi, J., Kato, J., Sasaki, K., Fujii, S., Watanabe, N., and Niitsu, Y. (1995). Loss of E-cadherin-dependent cell-cell adhesion due to mutation of the beta-catenin gene in a human cancer cell line, HSC-39. Mol Cell Biol 15, 1175-1181.

Kockel, L., Homsy, J. G., and Bohmann, D. (2001). Drosophila AP-1: lessons from an invertebrate. Oncogene 20, 2347-2364.

Lee, T., and Luo, L. (1999). Mosaic analysis with a repressible cell marker for studies of gene function in neuronal morphogenesis. Neuron 22, 451-461.

Leulier, F., Vidal, S., Saigo, K., Ueda, R., and Lemaitre, B. (2002). Inducible expression of double-stranded RNA reveals a role for dFADD in the regulation of the antibacterial response in Drosophila adults. Curr Biol 12, 996-1000.

Macara, I. G. (2004). Parsing the polarity code. Nat Rev Mol Cell Biol 5, 220-231. Malumbres, M., and Barbacid, M. (2003). RAS oncogenes: the first 30 years. Nat Rev Cancer 3, 459-465.

Martin-Blanco, E., Gampel, A., Ring, J., Virdee, K., Kirov, N., Tolkovsky, A. M., and Martinez-Arias, A. (1998). puckered encodes a phosphatase that mediates a feedback loop regulating JNK activity during dorsal closure in Drosophila. Genes Dev 12, 557-570.

Mihaly, J., Kockel, L., Gaengel, K., Weber, U., Bohmann, D., and Mlodzik, M. (2001). The role of the Drosophila TAK homologue dTAK during development. Mech Dev 102, 67-79.

Moreno, E., Basler, K., and Morata, G. (2002a). Cells compete for decapentaplegic survival factor to prevent apoptosis in Drosophila wing development. Nature 416, 755-759.

Moreno, E., Yan, M., and Basler, K. (2002b). Evolution of TNF signaling mechanisms: JNK-dependent apoptosis triggered by Eiger, the Drosophila homolog of the TNF superfamily. Curr Biol 12, 1263-1268.

Newsome, T. P., Asling, B., and Dickson, B. J. (2000). Analysis of Drosophila photoreceptor axon guidance in eye-specific mosaics. Development 127, 851-860.

Oyama, T., Kanai, Y., Ochiai, A., Akimoto, S., Oda, T., Yanagihara, K., Nagafuchi, A., Tsukita, S., Shibamoto, S., Ito, F., and et al. (1994). A truncated beta-catenin disrupts the interaction between E-cadherin and alpha-catenin: a cause of loss of intercellular adhesiveness in human cancer cell lines. Cancer Res 54, 6282-6287.

Pagliarini, R. A., Quinones, A. T., and Xu, T. (2003). Analyzing the function of tumor suppressor genes using a Drosophila model. Methods Mol Biol 223, 349-382.

Pagliarini, R. A., and Xu, T. (2003). A genetic screen in Drosophila for metastatic behavior. Science 302, 1227-1231.

Peifer, M., Rauskolb, C., Williams, M., Riggleman, B., and Wieschaus, E. (1991). The segment polarity gene armadillo interacts with the wingless signaling pathway in both embryonic and adult pattern formation. Development 111, 1029-1043.

Potter, C. J., Turenchalk, G. S., and Xu, T. (2000). Drosophila in cancer research. An expanding role. Trends Genet 16, 33-39.

Pulverer, B. J., Kyriakis, J. M., Avruch, J., Nikolakaki, E., and Woodgett, J. R. (1991). Phosphorylation of c-jun mediated by MAP kinases. Nature 353, 670-674.

Smeal, T., Binetruy, B., Mercola, D. A., Birrer, M., and Karin, M. (1991). Oncogenic and transcriptional cooperation with Ha-Ras requires phosphorylation of c-Jun on serines 63 and 73. Nature 354, 494-496.

Tao, W., Zhang, S., Turenchalk, G. S., Stewart, R. A., St John, M. A., Chen, W., and Xu, T. (1999). Human homologue of the Drosophila melanogaster lats tumour suppressor modulates CDC2 activity. Nat Genet 21, 177-181.

Varfolomeev, E. E., and Ashkenazi, A. (2004). Tumor necrosis factor: an apoptosis JuNKie? Cell 116, 491-497.

Wijnhoven, B. P., Dinjens, W. N., and Pignatelli, M. (2000). E-cadherin-catenin cell-cell adhesion complex and human cancer. Br J Surg 87, 992-1005.

Wodarz, A. (2000). Tumor suppressors: linking cell polarity and growth control. Curr Biol 10, R624-626.

Wu, H., and Arron, J. R. (2003). TRAF6, a molecular bridge spanning adaptive immunity, innate immunity and osteoimmunology. Bioessays 25, 1096-1105.

Xia, Y., and Karin, M. (2004). The control of cell motility and epithelial morphogenesis by Jun kinases. Trends Cell Biol 14, 94-101.

Xu, T., and Harrison, S. D. (1994). Mosaic analysis using FLP recombinase. Methods Cell Biol 44, 655-681.

Xu, T., and Rubin, G. M. (1993). Analysis of genetic mosaics in developing and adult Drosophila tissues. Development 117, 1223-1237.

Xu, T., Wang, W., Zhang, S., Stewart, R. A., and Yu, W. (1995). Identifying tumor suppressors in genetic mosaics: the Drosophila lats gene encodes a putative protein kinase. Development 121, 1053-1063.

Zhang, J. Y., Green, C. L., Tao, S., and Khavari, P. A. (2004). NF-kappaB RelA opposes epidermal proliferation driven by TNFR1 and JNK. Genes Dev 18, 17-22.

### EXAMPLE 3

### Validation in Human Cancer Cells

To validate the results obtained in Drosophila, the role of Ras and the human scribble homolog were evaluated in human cancer cells. We randomly selected human pancreatic cell lines having oncogenic Ras mutations and transplanted the cells into nude mice. Transplants of cell lines PaCa-2, AsPC-1 and Panc-1 developed metastatic tumors, while a transplant with cell line Capan-2 did not. We then evaluated protein levels of human Scrib in the cell lines. We found that in two out of three metastatic cell lines (PaCa-2, Panc-1) a truncated form of Scrib was expressed, suggesting an abnormality in the Scrib gene. In the third metastatic cell line, it was unclear whether a truncated form of Scrib was produced. In the Capan-2 cell line, only a single full-length Scrib protein was detected. Thus, it appears that in Ras mutant pancreatic cancer cell lines, metastatic behavior is associated with mutations in the Scrib cell polarity mutation. This result is consistent with the relationship between Scrib and Ras seen Drosophila, and indicates that the experiments conducted in Drosophila are effective in elucidating regulators of metastatic cell behavior that are generalizable to mammals, including humans.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

We claim:

1. A method for identifying a mutation that induces metastatic behavior in cells, the method comprising:
(a) providing a non-human transgenic animal in which cells comprise
(i) a genotype that induces non-invasive tumor formation and,
(ii) a candidate mutation;
(b) evaluating a metastatic behavior in the animal,
wherein an increase in the metastatic behavior of the cells in the animal relative to a suitable control indicates that the test mutation increases metastatic behavior.

2. The method of claim 1, wherein the genotype that induces the formation of a noninvasive tumor is restricted to a subset of cells in the animal.

3. The method of claim 1, wherein the genotype that induces noninvasive tumor formation is selected from the group consisting of: an oncogene and a loss of function mutation in a tumor suppressor gene.

4. The method of claim 3, wherein the oncogene is oncogenic Ras.

5. The method of claim 3, wherein the genotype includes a Ras^{V12} mutation.

6. The method of claim 3, wherein the mutation that induces noninvasive tumor formation is a loss of function mutation in the lats gene.

7. The method of claim 6, wherein the genotype includes a homozygous loss of function of the lats gene.

8. The method of claim 1, wherein the transgenic animal is a transgenic fly.

9. The method of claim 8, wherein the transgenic fly is Drosophila.

10. The method of claim 1, wherein the transgenic animal is a transgenic mouse.

11. The method of claim 1, wherein the candidate mutation is situated on an FLP chromosome.

12. A method for identifying a mutation that induces metastatic behavior in human cells, the method comprising:
(a) providing human cells which comprise:
(i) oncogenic Ras, and
(ii) a candidate mutation;
(b) evaluating a metastatic behavior in the cells,
wherein an increase in the metastatic behavior of the cells relative to a suitable control indicates that the test mutation increases metastatic behavior.

13. The method of claim 12, wherein (b) comprises transplanting the human cells into a suitable mouse host and determining whether metastasis occurs, wherein if metastasis occurs, a mutation that induces metastatic behavior has been identified.

14. A non-human transgenic animal whose cells comprise:
(a) an oncogene that induces non-invasive tumor formation and,
(b) a loss of function mutation in a cell polarity determining gene;
wherein the non-human transgenic animal develops metastatic tumors.

15. The non-human transgenic animal of claim 14, wherein the oncogene occurs in a subset of cells in the animal.

16. The non-human transgenic animal of claim 15, wherein the oncogene is Ras.

17. A non-human transgenic animal whose cells comprise:
(a) a loss of function mutation in a tumor suppressor gene and,
(b) a loss of function mutation in a cell polarity determining gene;
wherein the non-human transgenic animal develops metastatic tumors.

18. The non-human transgenic animal of claim 17, wherein the loss of function mutation in a tumor suppressor gene occurs in a subset of cells in the animal.

19. The non-human transgenic animal of claim 14 or 17, wherein the animal is selected from the group consisting of: a fly and a mouse.

20. The non-human transgenic animal of claim 14 or 17, wherein the cell polarity determining gene is selected from among the following: scribble (D. melanogaster), Scrib (human), Scrib1 (mouse), Lgl (D. melanogaster), Hub1 (human), Lgl1 (mouse), Dlg (Drosophila, mouse, human), Cdc42 (Drosophila, human, mouse), bazooka, stardust, cdc42 and another mammalian homolog of any of the preceding.

21. A method of screening for an inhibitor of metastatic behavior in cells, comprising:
(a) administering a candidate inhibitor to a test transgenic animal of claim 14 or 17, and
(b) evaluating metastatic behavior of cells in the test animal,
wherein a decrease in metastatic behavior of cells in the test transgenic animal indicates that the candidate inhibitor is an inhibitor of metastatic behavior in cells.

22. A method of screening for an inhibitor of metastatic behavior in cells, comprising:
(a) administering a candidate inhibitor to metastatic cells, wherein the metastatic cells comprise:
(i) an oncogene that induces non-invasive tumor formation and,
(ii) a loss of function mutation in a cell polarity determining gene;
(b) evaluating a metastatic behavior in the metastatic cells,
wherein a decrease in the metastatic behavior of the cells relative to a suitable control indicates that the candidate inhibitor decreases metastatic behavior in cells.

23. A method of screening for an inhibitor of metastatic behavior in cells, comprising:
(a) providing a non-human animal comprising transplanted human metastatic cells, wherein the metastatic cells comprise:
(i) an oncogene that induces non-invasive tumor formation and,
(ii) a loss of function mutation in a cell polarity determining gene;
(b) administering a candidate inhibitor to the non-human animal; and
(c) evaluating a metastatic behavior in the cells,
wherein a decrease in the metastatic behavior of the cells relative to a suitable control indicates that the candidate inhibitor decreases metastatic behavior.

24. The method of claim 23, wherein the non-human animal is a mouse.

25. A method of identifying an agent that may be used to inhibit metastatic cancer growth in a subject, the method comprising:
(a) identifying an agent that inhibits JNK pathway activation;
(b) evaluating the effect of the agent on a metastatic behavior of a metastatic cell,
wherein an inhibitor of JNK pathway activation that inhibits metastatic behavior of a metastatic cell is an agent that may be used to inhibit metastatic cancer growth in a subject.

26. The method of claim 25, wherein identifying an agent that inhibits JNK pathway activation comprises identifying an agent that inhibits the kinase activity of a kinase selected from the group consisting of: a JNKKK, a JNKK and a JNK.

27. The method of claim 26, wherein the JNKKK is a TAK1 kinase of human, mouse or Drosophila.

28. The method of claim 25, wherein identifying an agent that inhibits JNK pathway activation comprises identifying an agent that inhibits TNF-alpha signaling.

29. A method of identifying an agent that may be used to inhibit metastatic cancer growth in a subject, the method comprising:
(a) identifying an agent that activates a cell polarity protein;
(b) evaluating the effect of the agent on a metastatic behavior of a metastatic cell,
wherein an activator of a cell polarity protein that inhibits metastatic behavior of a metastatic cell is an agent that may be used to inhibit metastatic cancer growth in a subject.

30. The method of claim 29, wherein the cell polarity protein is selected from the group consisting of: scribble (D. melanogaster), Scrib (human), Scrib1 (mouse), Lgl (D. melanogaster), Hub1 (human), Lgl1 (mouse), Dlg (Drosophila, mouse, human), Cdc42 (Drosophila, human, mouse), bazooka, stardust, cdc42 and another mammalian homolog of any of the preceding.

31. A method of identifying an agent that may be used to inhibit metastatic cancer growth in a subject, the method comprising:
(a) identifying an agent that activates an E-cadherin pathway;
(b) evaluating the effect of the agent on a metastatic behavior of a metastatic cell,
wherein an activator of an E-cadherin pathway that inhibits metastatic behavior of a metastatic cell is an agent that may be used to inhibit metastatic cancer growth in a subject.

32. The method of claim 31, wherein activating an E-cadherin pathway comprises activating or upregulating a protein selected from the group consisting of: E-cadherin, armadillo, beta-catenin and another mammalian homolog of any of the preceding.
